(19) United States
(12) Patent Application Publication
(10) Pub. No.: US 2006/0127318 A1
(43) Pub. Date: Jun. 15, 2006

(54) Composition and method of a topical treatment of neurodermatitis

(75) Inventor: Rainer K. Liedtke, Gruenwald b. Muenchen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BIONICS PHARMA GmbH, Gruenwald b. Muenchen (DE)

(21) Appl. No.: 11/272,881

(22) Filed: Nov. 15, 2005

(30) Foreign Application Priority Data
Dec. 10, 2004 (DE) 10 2004 059 613.1

(51) Int. Cl. A61L 9/04; A61K 31/24

(52) U.S. Cl. 424/45; 514/534; 514/304

(57) ABSTRACT

A composition for the topical treatment of dermal irritation and pain symptoms of neurodermatitis comprising a topical pharmaceutical formulation which contains a therapeutically appropriate dose of a sodium channel blocker selected from the group of ester- or amide-type local anesthetics, wherein the sodium channel blocker is released directly on or in the skin underneath the applied pharmaceutical formulation.

The invention concerns the method and composition of a topical treatment of dermal irritation and pain symptoms of neurodermatitis by means of pharmaceutical formulations administered via the skin.

Neurodermatitis is a usually intermittently occurring inflammatory reaction of the skin with a chronic character. It is one of the so-called atopic diseases in which the endogenous immune system produces strong or inadequate defensive reactions against substances from the environment, so-called allergens. But psychological factors can also bring on or intensify episodes. The name is derived from a combination of the words neuron (nerve) and derma (skin) as well as the ending-itis for inflammation. Neurodermatitis often already begins in early childhood. The affected skin appears drier than normal skin and has a reduced capacity to store moisture and function as a mechanical barrier against environmental substances. Furthermore, the regulation of other skin functions like blood supply, hidropoiesis, and temperature is usually disturbed. This causes the skin to become chapped and rough, show a tendency to desquamation, and therefore also be clearly more sensitive to chemical or mechanical stimuli. We regard secondary stimulating effects on the peripheral neuronal pain conduction system as being the cause, which can explain that depending on the degree of severity, the skin irritations can range widely from mild irritation symptoms to very touch-sensitive pain, that is to say pain even in response to stimuli that are not normally painful (allodynia). Therefore, this also represents a great emotional strain.

With regard to the expression dermal irritation and pain symptoms, it should therefore also be mentioned that in the following context it always refers to those diseases that produce subjectively negative skin sensations ranging from mixed unpleasant irritation to pure pain.

Since until now no single name for the disease could be agreed upon, there are still numerous synonymous terms for it like, for example, atopic dermatitis, atopic eczema, endogenous eczema, and allergic eczema The frequently used synonym “atopic dermatitis” is meant to express that the disease episodes may also occur without a detectable external cause. In this connection, “allergic dermatoses” means skin changes that are caused by allergens. In the following context, therefore, the functionally subordinate term neurodermatitis always also means and includes these synonymous terms.

According to the present point of view, the primary cause of neurodermatitis is an excessive defensive reaction of the body to exogenous substances (e.g. from food). But the disease can also be caused or aggravated by mechanical irritations of the skin, infections, or climatic conditions. In the course of this disease, special subgroups of the white blood cells, lymphocytes, are directed against these allergens. This leads to the formation of special defensive substances, antibodies. Via interactions with various messenger substances of the immune system, so-called cytokines, a defensive inflammatory reaction of the skin then occurs. This is maintained by various white blood cells (lymphocytes). In this connection, inflammatory substances that further intensify the defensive reaction and its main symptoms are released.

Due to its everyday causes, the prevalence and incidence rate in the general population are high. Neurodermatitis is therefore one of the most common skin diseases; children are particularly commonly affected.

Pharmacologically speaking, at present no treatment that can be regarded as the universally applicable standard exists; the treatment is usually carried out empirically and according to the individual conditions. Irritative and painful skin symptoms of neurodermatitis are very often treated first with topical pharmaceutical formulations containing cortisone, otherwise with formulations containing ingredients like, for example, zinc, certain oils, antihistamines, or vegetable components, and recently also increasingly with immunosuppressants in ointment form. In many cases, above all in long-term cases, the treatment involves only active agent-free ointments or gels which are aimed at reducing the skin irritation. If infectious causes or their involvement are suspected, topical or systemic antibiotics are also administered. At present, it does not yet seem possible to cure the disease by means of a causal treatment. All in all, the current symptomatic treatments are therefore usually prolonged, partly with low response rates, and in many individual cases also not successful. Moreover, systemic treatments (e.g. with cortisone) involve significant side effects. Hence, numerous attempts with so-called alternative treatments are made in this area.

With respect to the mode of administration, the expression “topical application” contrasts with that of the systemic application of medicinal products. In the text that follows, topical application means the local administration of medicinal agents. In the treatment of skin irritation symptoms and pain, this therefore means the administration of the medicinal agent directly on the irritated or painful skin region. This can be the case with an ointment or a gel, for example. Consequently, the site of action is largely identical with the site of administration. This contrasts with systemic administration via which a medicinal agent, a tablet for example, is first distributed throughout the entire body by the blood stream after absorption in the stomach and intestines, and only afterwards does a small fraction reach the site of the painful cause. Accordingly, the site of administration and site of action are not identical with systemic treatments. Hence, all systemic treatments have a higher rate of undesirable effects. The expressions used here like “topical form of administration” or “topical composition” therefore mean pharmaceutical preparations that are made up for the purpose of topical application.

Within the complex cascade of the cellular pain mechanism, the biochemical tissue inflammatory processes resulting from allergic reactions play an important role, especially the formation of prostaglandins. These are hormone-like substances which are found in chemically different forms throughout the human body and play a role in the development of inflammatory processes. In the course of inflammations, they increase among other things the sensitivity of pain receptors and increase the release of various local algesic mediators and formation of local edemas.

Local anesthetics are generally used in medicine as efficacious local anesthetic agents (e.g. during operations), and some substances from this class are also employed systemically to stabilize the cardiac rhythm. In pain therapy, local injections with local anesthetics have also proven to be successful in some indications. This was based on the concept of interrupting the functioning of nerve tracts by anesthetizing them. As another pharmacological principle here, a noninvasive administration of local anesthetics with topical plaster systems has also proven its value for the treatment of neuromuscular pain. This new therapy was first invented and introduced by us in recent years and carried out with U.S. Pat. No. 5,776,952 for the area of topical administration of local anesthetics for back pain, and with U.S. Pat. No. 5,840,755 for headache. In this connection, it was also shown that local anesthetics are not only clinically applicable as conventional anesthetics but also as specific, neurally acting analgesics with neuromuscular pain.

Amide- or ester-type local anesthetics (e.g. amide-type lidocaine) show as a pharmacological mechanism of action an inhibition of the rapid sodium ion influx into nerve fibers; that is, they act as so-called sodium channel blockers. Via this specific effect, they block the impulse conduction in nerve fibers, which concerns in principle all regional nerve fibers. However, since the sensory pain-conducting fibers (unmyelinated C fibers and myelinated Aδ fibers) are anatomically thinner than the motor fibers (e.g. Strichartz, G. R. [ed.]: Local Anesthetics, Handbook of Experimental Pharmacology, Vol. 81. Springer, Berlin-N.Y. 1987), different effects can be produced via the topically administered dose. In this connection, it also seems to be of therapeutic relevance that the thickness of the peripheral B fibers of the autonomic nervous system is between that of the C and Aδ fibers. An additional assumption for a lower-dose transcutaneous therapeutic approach was to regard afferent bioelectric pain signals as irregular neuronal information patterns which can be regularized via lower local anesthetic doses. These predictions and implicit assumptions from among others U.S. Pat. No. 5,776,952 and U.S. Pat. No. 5,840,755 of an effect of lidocaine on ectopic impulses was experimentally verified in recent years (Khodorova A., Meissner K., Leeson S., Strichartz G. R.: Muscle Nerve. 24; 634 [2001], Persaud N., Strichartz G. R. Pain. 99, 333 [2002]).

Therefore, a peripheral topical transcutaneous pain reduction requires lower doses of local anesthetics than those with which an anesthesia of neural function is produced. Therefore, a transcutaneous analgesia can also be distinguished by its selectivity on pain receptors from a transcutaneous anesthesia, since the latter affects all local receptors and consequently also causes an inactivation of motor functions, which is undesirable in the case of the therapeutic aim of only analgesia.

All in all, it therefore turns out that the dermal irritation- and pain-reducing mechanism of action of local anesthetic-type sodium channel blockers is exerted on the cellular level at different points in the peripheral local pain cascade. They show their specific effects relevant for pain reduction only on the level of the reactively, that is secondarily, developing neuronal processes of conduction of pain signals resulting from an inflammation. In the context of the cellular process of skin inflammation with neurodermatitis, which essentially also influences the unmyelinated C fibers and myelinated Aδ fibers, it can be concluded that irritation and pain symptoms can be rapidly affected there by a topical administration of a local anesthetic-type sodium channel blocker. Therapeutically, a local administration of these substances should therefore result in a rapid effect on skin irritation and skin pain and the related or associated symptoms. Furthermore, a normalization of local neuronal irritation can also result in a local normalization of the interlinked regulatory mechanisms of skin functions like blood supply, hidropoiesis, and temperature. These functional disturbances lead also to trophic-morphological disturbances and thereby continue to maintain the skin irritation and pain.

The aim of the invention is to improve the dermal irritation and pain symptoms of neurodermatitis with a topical therapy via the skin.

This aim is achieved by using a topical pharmaceutical formulation suitable for the skin, which contains a therapeutically appropriate dose of a sodium channel blocker from the class of ester- or amide-type local anesthetics, that releases the substances directly on or in the skin regions beneath the pharmaceutical formulation.

To increase the possibilities for application of the therapy, in a further development of the invention the topical pharmaceutical formulation is an ointment, a gel, an emulsion, suspension or lotion, a solution or a spray.

To specify a suitable therapy, lidocaine, a sodium channel blocker from the class of amide-type local anesthetics, is used as an active agent in the topical pharmaceutical formulation; the concentration of this substance can range from 0.1% to 20%.

To improve the efficacy and tolerance of the treatment, another sodium channel blocker from the class of amid- or ester-type local anesthetics is used in the topical pharmaceutical formulation—tetracaine, prilocaine, bupivacaine, mepivacaine, etidocaine, procaine, benzocaine, propoxycaine, hydroxyprocaine, chloroprocaine, ambucaine, metabutoxycaine, proparacaine, paraethoxycaine, butacaine, isobucaine, hexylcaine, piridocaine, piperocaine, cyclomethycaine, procainamide, dibucaine, pyrrocaine, or tolycaine; the concentration of this substance can range from 0.1% to 20%.

To increase the possibilities for medical application of the therapy, the topical pharmaceutical formulation is used for the treatment of painful or initation-induced skin symptoms, which are based on primary or secondary local neural irritation, including symptoms that occur in the skin diseases of psoriasis, rosacea, perioral dermatitis, lichen ruber, stasis ulcer of the leg, and allergic dermatoses.

To increase the possibility for medical application of the therapy, the topical pharmaceutical formulation is used for medical purposes in both the field of human medicine and the field of veterinary medicine.

A treatment of neurodermatitis, especially its dermal irritation and pain symptoms, with topical pharmaceutical formulations containing a sodium channel blocker from the class of ester- or amide-type local anesthetics has not been previously described or used. This doesn't seem surprising because both its pharmacological action profile and its previous practical medical use are in other indications. Without an analysis of the cellular sequence of events in the local pain process in neurodermatitis, including the inflammatory processes produced there as well as its resulting trophic and morphological sequelae, the usefulness of this substance class for neurodermatitis is not discernible or obvious. Only this analysis shows that a topical treatment with sodium channel blockers causes a reduction of irritation and pain via the regulation of the reactive irritated sensory neuronal conduction of the pain transmission. Thus, the main subjective symptoms of neurodermatitis, skin irritation and pain, are caused as reaction to the skin inflammation via polymodal neuronal nociceptors, particularly unmyelinated C fibers and Aδ fibers. Also many of the chemical substances involved in the inflammation like, for example, biogenic amines, histamine, serotonin, proteases, and neuropeptides can indirectly or directly activate the C fibers and lead in this way to stimulation with neural irritation. Mechanical influences can intensify this sensory stimulation. It is possible to influence it with sodium channel blockers, and they then result in a rapid reduction of the dermal irritation and pain symptoms. By means of this primary pharmacological effect on neuronal transmission, a regulation of associated local vegetative skin functions like blood supply, hidropoiesis, and temperature also occurs mainly as secondarily effects. We assume this is due to an indirect α-receptor-blocking effect of the sodium channel blockers on the cutaneous vessels that takes place via the local connection of the afferent C and Aδ fibers with the postganglionic sympathetic B fibers in the spinal dorsal root. Such a vasotropic effect, resulting in improved local blood circulation, can then produce a further normalization of trophic disturbances and their visible morphological sequelae on the skin. Dose-dependent direct effects of the sodium channel blockers on the peripheral Aα fibers of the cutaneous blood vessels may also occur. However, these effects of the sodium channel blockers concern only the local reactions, since those that are produced via the second vegetative reaction route (i.e. those central hormonal reactions produced via the hypothalamic-adrenal stress axis) are not affected by them.

The overall effect also cannot be achieved by a systemic administration of these substances. It can only be adequately produced if the intervention takes place with a suitable dosage directly on the site of irritation and pain-inducing lesion, which cannot be adequately controlled with a systemic dosage. Furthermore, a systemic administration would involve a high risk of unspecific damage. Other than with the very safe topical administration, the systemic administration of a local anesthetic would involve a high toxic risk (e.g. due to its cardiovascular effects).

All in all, the treatment therefore has the advantage of an improved and rapid reduction of the clinical symptoms, especially the dermal irritation and pain symptoms which are always most crucial for patents. This results via regularization of the terminal afferent cutaneous nerves irritated by inflammation, which also leads secondarily to an improvement of the trophic and morphological skin condition. This therapeutic effect which extends from the primary elimination of neuronal irritation symptoms, via a retrograde chain reaction, to morphological improvements therefore even shows signs of a curative effect. This cannot be achieved at the present time with any other treatment, particularly not in this short period of time.

### EXAMPLE 1

A general illustrative example of the form of a topical pharmaceutical formulation, however without wanting to limit it to this, can be a gel formulation familiar to the pharmaceutical expert in which 20 milligrams lidocaine, as a hydrochloride, is contained in 1 gram of this gel as an amide-type local anesthetic sodium channel blocker. It is therefore a skin gel containing 2% of the active agent. Depending on the size of the area, approx. 0.5-2 grams of this gel can be thinly applied on the neurodermatitis-induced irritated or painful skin area up to 3 times daily. During the further course of treatment, the dose can be gradually reduced and the gel only needs to be dabbed onto the remaining irritated areas as needed.

A rapid onset of efficacy was already observed in the first individual medical cases in which irritating and painful skin symptoms on parts of the face that had persisted for some time in spite of a previous neutral ointment treatment then showed a significant improvement of these agonizing symptoms within only a few minutes after treatment with a topical formulation corresponding to that in example 1. Furthermore, the micropapular eruptions had already declined within the next few hours and then later a fading away of the reddening on inflamed skin areas occurred, with smoothing of the skin surface and visible normalization of the cutaneous circulation. No other therapeutic measures were required.

1. Method and composition of a topical treatment of dermal irritation and pain symptoms of neurodermatitis, comprising: a topical pharmaceutical formulation suitable for the skin, which contains a therapeutically appropriate dose of a sodium channel blocker from the class of ester- or amide-type local anesthetics, is used, wherein the contained substance is released directly on or in the skin region underneath the pharmaceutical formulation.

2. Method and composition as recited in the previous claim, wherein the topical pharmaceutical formulation is an ointment, a gel, an emulsion, suspension or lotion, a solution or a spray.

3. Method and composition as recited in the previous claims, wherein lidocaine, a sodium channel blocker from the class of amide-type local anesthetics, is used as the active agent; the concentration of this substance can range from 0.1% to 20%.

4. Method and composition as recited in the previous claims, wherein tetracaine, prilocaine, bupivacaine, mepivacaine, etidocaine, procaine, benzocaine, propoxycaine, hydroxyprocaine, chloroprocaine, ambucaine, metabutoxycaine, proparacaine, paraethoxycaine, butacaine, isobucaine, hexylcaine, piridocaine, piperocaine, cyclomethycaine, procainamide, dibucaine, pyrrocaine, or tolycaine is used as another sodium channel blocker from the class of amid- or ester-type local anesthetics; the concentration of this substance can range from 0.1% to 20%.

5. Method and composition as recited in the previous claims, wherein the topical pharmaceutical formulation is also used for the treatment of irritative or painful skin symptoms, which are based on primary or secondary local cutaneous neural irritation, including symptoms that occur in the skin diseases of psoriasis, rosacea, perioral dermatitis, lichen ruber, stasis ulcer of the leg, and allergic dermatoses.

6. Method and composition as recited in the previous claims, wherein the topical pharmaceutical formulation is used for medical purposes both in the field of human medicine and in the field of veterinary medicine.
